(19) United States
(12) Patent Application Publication
(10) Pub. No.: US 2003/0087798 A1
(43) Pub. Date: May 8, 2003

(54) Atopy-associated sequence variants on chromosome 12

(76) Inventors: Benjamin Raby, Boston, MA (US); Thomas J. Hudson, Montreal (CA); Catherine Laprise, Chicoutimi (CA)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/152,326

(22) Filed: May 20, 2002

Related U.S. Application Data
(60) Provisional application No. 60/292,047, filed on May 18, 2001.

(51) Int. Cl. A61K 31/00; C12Q 1/68; G01N 33/567

(52) U.S. Cl. 514/1; 435/6; 435/7.21

(57) ABSTRACT

Methods and kits for determining an individual's susceptibility to atopy-associated disease are described. Also described are nucleic acids comprising alleles linked to atopy-associated diseases.

### RELATED APPLICATION

This application claims the benefit of U.S. Provisional Application No. 60/292,047, filed on May 18, 2001.

The entire teachings of the above application are incorporated herein by reference.

### BACKGROUND OF THE INVENTION

Asthma, or Reversible Obstructive Airway Disease (ROAD), is a condition in which the airways of the lungs become either narrowed or completely blocked, impeding normal breathing and potentially leading to more severe health problems. Although normal airways have the potential for constricting in response to allergens or irritants, the asthmatic's airways are oversensitive or hyper-reactive. In response to stimuli, the airways may become obstructed by one or more of the following: constriction of the muscles surrounding the airway; inflammation and swelling of the airway; and increased mucus production that clogs the airway. Once the airways have become obstructed, it takes more effort to force air through them, and breathing becomes labored. Because exhaling through the obstructed airways is difficult, stale air accumulates in the lungs and decreases the amount of fresh air that can be taken in with each new breath. Thus, not only is there less oxygen available for the whole body, the high concentration of carbon dioxide in the lungs causes the blood supply to become acidic as well. This acidity in the blood may rise to toxic levels if the asthma remains untreated.

Although asthma creates difficulties in breathing and can lead to more serious problems, the lung obstruction associated with asthma is reversible, either spontaneously or with medication. Treatments for asthmatics include anti-inflammatory agents such as corticosteroids, bronchodilators and leukotriene antagonists.

### SUMMARY OF THE INVENTION

The present invention relies on the unexpected finding that a particular genetic locus, chromosome 12 at marker D12S1052, is genetically linked to atopy-associated (i.e., hyperresponsive) diseases such as, for example, asthma, allergic rhinitis and atopic dermatitis. This particular region was mapped in detail, and a total of 20 polymorphisms in 7 genes were identified as being linked to atopy-associated diseases.

In one embodiment, the invention is directed to a method for determining an individual's susceptibility to atopy-associated disease, comprising detecting in a biological sample obtained from the individual an allele at a polymorphic site in the genetic region defined by the D12S376*152-D12S828*291-D12S869*226 haplotype wherein said allele is identified as being linked to the atopy-associated disease phenotype. In a particular embodiment, the atopy-associated disease is asthma. In one embodiment, the method of detecting the allele comprises using an allele-specific indicator. In another embodiment, the method of detecting the allele comprises a hybridization assay, and the allele-specific indicator is one or more allele-specific hybridization probe. The one or more allele-specific hybridization probes can be contained in a microarray. In another embodiment, the allele-specific indicator is an allele-specific antibody.

In one embodiment, the invention id directed to a method for determining an individual's susceptibility to atopy-associated disease, comprising detecting in a biological sample obtained from the individual one or more alleles at one or more polymorphic sites, said one or more alleles selected from the group consisting of: NM_{—}004537 c206+6 G>A, NM_{—}004537 c206+14 A>G, NM_{—}004537 c206+105 T>C, NM_{—}004537 c300 C>T, NM_{—}004537c348+45 A>G, NM_{—}004537 c348+57 C>T, NM_{—}004537 c429+39 C>T, NM_{—}004537 c471 A>G, NM_{—}001874 c258-95 C>A, NM_{—}001874 c837 G>A, NM_{—}001874 c1077 T>C, NM_{—}001874 c1758 T>C, NM_{—}014505 c336+70 A>G, NM_{—}000899 c628 G>T, NM_{—}007199 c133-71 T>A, NM_{—}007199 c435 A>G, NM_{—}007199 c439 G>A, NM_{—}003153 c1089+29 G>A, and POCHA c321 C>T, and wherein detection of the allele is indicative of the individual's susceptibility to atopy-associated disease. In a particular embodiment, the individual is of French-Canadian descent. In one embodiment, an allele-specific antibody can be used to detect NM_{—}000899 c628 G>T or NM_{—}007199 c439 G>A.

In another embodiment, the invention is directed to a method for determining an individual's susceptibility to atopy-associated disease, comprising detecting the D12S376*152-D12S828*291-D12S869*226 haplotype, wherein the presence of the haplotype is indicative of an increased susceptibility to atopy-associated disease.

In another embodiment, the invention is directed to a method for determining an individual's susceptibility to atopy-associated disease, comprising detecting the D12S828*291-D12S869*226 haplotype, wherein the presence of the haplotype is indicative of an increased susceptibility to atopy-associated disease.

In another embodiment, the invention is directed to a nucleic acid molecule comprising an allele selected from the group consisting of: NM_{—}004537 c206+6 G>A, NM_{—}004537 c206+14 A>G, NM_{—}004537 c206+105 T>C, NM_{—}004537 c348+45 A>G, NM_{—}004537 c348+57 C>T, NM_{—}004537 c429+39 C>T, NM_{—}001874 c258-95 C>A, NM_{—}001874 c837 G>A, NM_{—}001874 c1077 T>C, NM_{—}007199 c133-71 T>A, NM_{—}007199 c435 A>G, NM_{—}003153 c1089+29 G>A, and POCHA c321 C>T.

In yet another embodiment, the invention is directed to a kit for determining an individual's susceptibility to atopy-associated disease comprising reagents for determining one or more alleles at one or more polymorphic sites selected from the group consisting of: NM_{—}004537 c206+6, NM_{—}004537 c206+14, NM_{—}004537 c206+105, NM_{—}004537 c300, NM_{—}004537 c348+45, NM_{—}004537 c348+57, NM_{—}004537 c429+39, NM_{—}004537 c471, NM_{—}001874 c258-95, NM_{—}001874 c837, NM_{—}001874 c1077, NM_{—}001874 c1758, NM_{—}014505 c336+70, NM_{—}000899 c628, NM_{—}007199 c133-71, NM_{—}007199 c435, NM_{—}007199 c439, NM _{—003153} c1089+29, and POCHA c321. In a particular embodiment, the kit comprises an allele-specific indicator. The allele-specific indicator can be, for example, an allele-specific probe or an allele-specific antibody.

In another embodiment, the invention is directed to an oligonucleotide microarray having immobilized thereon a plurality of probes, wherein at least one of the probes is specific for an allele at a polymorphic site selected from the group consisting of: NM_{—}004537 c206+6, NM_{—}004537 c206+14, NM_{—}004537 c206+105, NM_{—004537} c300, NM_{—}004537 c348+45, NM_{—}004537 c348+57, NM_{—}004537 c429+39, NM_{—}004537 c471, NM_{—}001874 c258-95, NM_{—}001874 c837, NM_{—}001874 c1077, NM_{—}001874 c1758, NM_{—}014505 c336+70, NM_{—}000899 c628, NM_{—}007199 c133-71, NM_{—}007199 c435, NM_{—}007199 c439, NM_{—003153} c1089+29, and POCHA c321.

In yet another embodiment, the invention is directed to a method for determining an individual's susceptibility to atopy-associated disease comprising: obtaining a biological sample from the individual; and determining the amino acid present at one or more of amino acid position 147 of the Interleukin Receptor Associated Kinase and amino acid position 210 of Stem Cell Factor, wherein presence of one or more of an isoleucine at amino acid position 147 of the Interleukin Receptor Associated Kinase or a tyrosine at amino acid position 210 of Stem Cell Factor is indicative of increased likelihood of an atopy-associated disease in the individual as compared with an individual having a valine at amino acid position 147 of the Interleukin Receptor Associated Kinase or an aspartate at amino acid position 210 of Stem Cell Factor, respectively. In a particular embodiment, the amino acid present at both position 147 of the Interleukin Receptor Associated Kinase and position 210 of Stem Cell Factor is determined.

### DETAILED DESCRIPTION OF THE INVENTION

Asthma is a phenotypically heterogeneous disorder associated with intermittent respiratory symptoms such as, e.g., bronchial hyperresponsiveness and reversible airflow obstruction. One or more genetic components to asthma have been suggested by studies demonstrating increased prevalence of atopy-associated diseases in family and twin comparisons (Ober, C. et al., 1998. Hum. Mol. Genet., 7:1393-1398; Daniels, S. et al., 1996. Nature, 383:247-250; The CSGA, 1997. Nat. Genet., 15:389-392; Wjst, M. et al., 1999. Genomics, 58:1-8; Dizier, M. et al, 2000. Am. J Resp. Crit. Care Med., 162:1812-1818). Although these previous studies indicate a possible genetic component, such studies may also reflect racial anomalies or shared environmental exposures. Described herein is the first direct evidence demonstrating a genetic link between heritable genetic elements and atopy-associated diseases, e.g., asthma, allergic rhinitis and atopic dermatitis. Other atopy-associated phenotypes include, for example, allergic sensitization, elevated total serum IgE and eosinophilia.

The present invention relates to methods and compositions for characterization of “alleles” that are in “linkage disequilibrium” with an atopy-associated disease, e.g., asthma. As used herein, “allele” refers to a specific sequence variant possible at a polymorphic site. A “polymorphic site” is a position in a polynucleotide sequence that can have more than one possible allele. “Polymorphic” is a referential term that compares a sequence to at least one other sequence. For example, at a particular site on a chromosome or in a reference sequence, one individual in a population might have a guanine while another individual might have an adenine. Such a site is a polymorphic site having two different alleles at that site; one allele has a guanine at the polymorphic site, while the other allele has an adenine at the polymorphic site. Any sequence position can be a polymorphic site provided more than one possible allele occurs at the site.

As used herein, “linkage disequilibrium” refers to the inheritance of heritable elements (e.g., alleles, phenotypes, genotypes) in a manner that would not be expected statistically if such elements were inherited “randomly” (i.e., the chances of inheriting a particular heritable element are independent of whether or not a different heritable element is inherited). For example, when heritable elements are “linked,” there is an increased probability that the two heritable elements will be inherited together instead of inherited independently, e.g., a specific allele of a gene can be said to be in linkage disequilibrium with a specific phenotype if, in a population, the genotype of an individual displaying the phenotype is more likely to carry the particular allele than would be expected if the allele and phenotype were inherited independently of each other.

Alleles are randomly assorted or inherited independently if the frequency of the two alleles together is the product of the frequencies of the two alleles individually. For example, if two alleles at different polymorphic sites are present in 50% of the chromosomes in a population, then they would be said to assort randomly if the two alleles are present together on 25% of the chromosomes in the population. A statistically significant higher percentage would mean that the two alleles are linked. For example, a polymorphic site, “g.-50” (see below for an explanation of nomenclature), having two alleles, “g.-50A” and “g.-50C”—each allele being present in 50% of the individuals in a given population, is said to be in linkage disequilibrium with respect to another polymorphic site, “g.-75,” having two alleles, “g.-75G” and “g.-75T”—each each allele being present in 50% of the individuals in a given population, if particular combinations of alleles (e.g., g.-50A/g.-75G) are observed in individuals at a frequency that is statistically significantly greater than 25% (if the polymorphic sites are not linked, then one would expect a 50% chance of an individual having g.-50A and a 50% chance of having g.-75G—thus leading to a 25% chance of having the combination of g.-50A/g.-75G together). Heritable elements that are in linkage disequilibrium are said to be “linked” or “genetically linked” to each other.

A systematic nomenclature has been proposed for describing polymorphic sites and alleles. Sequence variations are described in relation to a reference sequence, e.g., sequences referenced by database accession numbers. The first letter (followed by a period), denotes the source of the sequence, e.g., “g.” denotes a genomic sequence, “c.” denotes a cDNA sequence, “m.” denotes a mitochondrial sequence, “r.” denotes an RNA sequence, and “p.” denotes a protein sequence. Following the source, a number corresponding to the first affected nucleotide in the reference sequence is followed by the reference nucleotide, the “>” symbol which denotes a substitution, and the variant nucleotide. For example, an adenine at position 76 in the reference sequence that has a variant thymidine at the position in the sequence to be named, would be referred to as “g.76A>T” when in reference to a genomic sequence. According to convention, nucleotide “+1” is the adenine of the ATG start codon; the nucleotide 5′ to +1 is numbered “−1”. There is no nucleotide corresponding to “0”. The nucleotide 3′ of the translation termination codon is “*1”. Intron sequences are designated by the nucleotide number corresponding to the last nucleotide of the preceding exon, a “+”, and the position in the intron of the affected nucleotide. For example, a G>T variant 35 nucleotides 3′ of the end of exon 1 (which occurs at nucleotide number 75 of the reference sequence), is designated, “g.75+35G>T”. Alternatively, if the exon number is known, the variant can be described as “g.IVS1+35G>T” since the variant position occurs 3′ from exon 1.

In particular, the present invention relates to, but is not limited to, single nucleotide polymorphisms (hereinafter, “SNP,” is used to refer to a polymorphic site that is a single nucleotide as opposed to several nucleotides in length, or, when a reference sequence is known, “SNP” can be used to refer to a specific allele at a single nucleotide polymorphic site), that are linked to atopy-associated diseases such as, for example, asthma, allergic rhinitis and atopic dermatitis, or other atopy-associated phenotypes such as, for example, allergic sensitization, elevated total serum IgE and eosinophilia. These SNPs are in linkage disequilibrium with atopy-associated diseases, as measured by quantifiable indices known in the art. In other words, a particular allele at a polymorphic site described herein is associated with atopy-associated diseases. As such, the SNPs described herein are useful as “genetic markers,” i.e., sequence elements that are indicative of other sequence elements or phenotypes, e.g., asthma or other atopy-associated diseases. The methods of the present invention are not limited to the use of SNPs as genetic markers, as other alleles representing larger polymorphic sites (e.g., substitutions, deletions, insertions or translocations that span more than a single nucleotide) can serve as genetic markers for atopy-associated diseases.

A search for sequence variants in and around chromosome 12 at marker D12S1052 led to the identification of twenty SNPs in a region defined by the D12S376*152-D12S828*291-D12S869*226 haplotype. These polymorphisms occurred in a region of seven genes (used herein to describe a DNA sequence that can be transcribed, including both coding and non-coding sequences). Seven of these SNPs have been previously reported, but association of these polymorphisms with atopy-associated diseases has not been previously reported.

In addition to their physical proximity to a locus linked to atopy-associated diseases, the polymorphisms described herein are linked to phenotypic indicators of atopy-associated diseases known in the art. The genetic disequilibrium described herein between the polymorphisms and atopy-associated diseases indicates that the polymorphisms described herein are useful as markers for atopy-associated diseases.

The invention is directed to methods for detecting alleles at polymorphic sites, such that the particular allele is indicative of an individual's susceptibility to atopy-associated disease. Disclosed herein as a genetic region defined by the D12S376*152-D12S828*291-D12S869*226 haplotype, which is linked to atopy-associated diseases. Specific alleles at polymorphic sites within this region are useful as indicators of atopy-associated disease. In addition, the absence of specific alleles is indicative of a decreased susceptibility to atopy-associated disease relative to an individual that carries the specific allele.

As several of the SNPs described herein are present in the coding sequences (used herein to refer to the DNA sequence that is actually transcribed) of genes, the SNPs described herein can lead to different gene products during transcription and translation (e.g., c628 G>T leads to a Asp210Tyr change, and c439 G>A leads to a Val147Ile change). The polymorphisms linked to atopy-associated diseases can have functional consequences for the expressed protein, and, therefore, these polymorphisms can be the target for therapeutic and drug discovery assays. Additionally or alternatively, these polymorphisms allow for the use of protein diagnostic methods to detect the presence or absence of a particular allele.

In general, the detection in a biological sample obtained from an individual of a particular allele at a polymorphic site that is genetically linked to a particular gene or phenotype, is indicative of a particular allele of the gene or of the presence of the particular phenotype. The identification of the genetic linkage between specific polymorphic sites and atopy-associated diseases described herein allows for the inference to be made that the detection of a particular allele in a sample is indicative that the individual from whom the sample was obtained also exhibits the linked phenotype, e.g., atopy-associated disease.

The sample to be assessed can be any sample that contains a gene expression product. Suitable sources of gene expression products, i.e., samples, can include cells, lysed cells, cellular material for determining gene expression (e.g., subcellular fractions), or material containing gene expression products. Examples of such samples are blood, plasma, lymph, urine, tissue, mucus, sputum, saliva, intestinal tissue or other cell samples. Methods of obtaining such samples are known in the art. For the purposes of the invention, individuals from whom samples are obtained can be, for example, human patients or other mammals. Such patients may or may not exhibit atopy-associated characteristics or phenotypes or diseases related to atopy. Additionally, samples can be obtained from humans who are undergoing treatment for atopy-associated diseases.

As used herein, “gene expression products” are proteins, polypeptides, or nucleic acid molecules (e.g., mRNA, tRNA, rRNA, cDNA, or cRNA) that result from transcription and/or translation of genes. The nucleic acid molecule levels measured can be derived directly from the gene or, alternatively, from a corresponding regulatory gene or regulatory sequence element. All forms of gene expression products can be measured by methods known in the art. For example, the nucleic acid molecule can be transcribed to obtain an RNA gene expression product. If desired, the transcript can be translated using, for example, in vitro translation methods to obtain a polypeptide gene expression product. Polypeptide gene expression products can be detected in protein binding assays, for example, antibody assays, or in nucleic acid binding assays, known in the art. Additionally, variants of genes and gene expression products including, for example, spliced variants and expression products translated or transcribed from polymorphic alleles, can be measured. Similarly, gene expression can be measured by assessing the level of a polypeptide or protein or derivative thereof translated from mRNA.

Methods are well known in the art for detection of alleles at specific polymorphic sites, including, for example, sequencing, PCR-based assays, hybridization assays, and, when applicable, allele-specific protein-detection methods. If the particular polymorphic site is in linkage disequilibrium with a particular phenotype, e.g., atopy-associated disease, then the detection of a specific allele is indicative of the particular phenotype. Thus, diagnostic tests can be performed quickly and accurately for any phenotypes that are genetically linked to allele(s) at the polymorphic site(s) described herein.

The invention further provides for the use of allele-specific indicators in order to determine the particular allele present in a sample. An allele-specific indicator can be, for example, a hybridization probe, a PCR primer, an allele-specific antibody, etc. Oligonucleotides (e.g., probes and primers) that hybridize to one or more allelic variants, or to their complementary sequences, are useful nucleic acids for detecting the presence of a particular allele in a sample. Such oligonucleotides will hybridize to one polymorphic form of the nucleic acid molecules described herein but not to the other polymorphic form(s) of the sequence, i.e., are allele-specific. Thus, such oligonucleotides can be used to determine the presence or absence of particular alleles of the polymorphic sequences described herein.

Hybridization probes are oligonucleotides that bind in a base-specific manner to a complementary strand of nucleic acid. Such probes include peptide nucleic acids (hereinafter, “PNA”), as described in Nielsen et al. (1991. Science 254, 1497-1500). Probes can be any length suitable for specific hybridization to the target nucleic acid sequence. The most appropriate length of the probe may vary depending upon the hybridization method in which it is being used; for example, particular lengths may be more appropriate for use in microfabricated arrays (microarrays), while other lengths may be more suitable for use in classical hybridization methods. Such optimizations are known to one of skill in the art. Suitable probes and primers can range from about 5 nucleotides to about 30 nucleotides in length. For example, probes and primers can be 5, 6, 8, 10, 12, 14, 16, 18, 20, 22, 24, 25, 26, 28 or 30 nucleotides in length. Additionally, a probe can be a genomic fragment that can range in size from about 25 to about 2,500 nucleotides in length. The probe or primer preferably overlaps at least one polymorphic site occupied by any of the possible variant nucleotides. The nucleotide sequence can correspond to the coding sequence of the allele or to the complement of the coding sequence of the allele.

Hybridization can be performed under stringent conditions, e.g., at a salt concentration of no more than 1 M and a temperature of at least 25° C. For example, conditions of 5×SSPE (750 mM NaCl, 50 mM Na-Phosphate, 5 mM EDTA, pH 7.4) and a temperature of 25-30° C., or equivalent conditions, are suitable for allele-specific probe hybridizations. Equivalent conditions can be determined by varying one or more of the parameters given as an example, as known in the art, while maintaining a similar degree of identity or similarity between the target nucleotide sequence and the primer or probe used.

Conditions for stringency can be as described in WO 98/40404, the teachings of which are incorporated herein by reference. In particular, examples of “highly stringent,” “stringent,” “reduced,” and “least stringent” conditions are provided in WO 98/40404 in the Table on page 36, which is reproduced below. Highly stringent conditions are those that are at least as stringent as, for example, conditions A-F; stringent conditions are at least as stringent as, for example, conditions G-L; and reduced stringency conditions are at least as stringent as, for example, conditions M-R.

For the purposes of the present invention, detection of SNPs will typically utilize highly stringent hybridization and wash conditions.

| | | Hybrid | | Wash |
|---|---|---|---|---|
| Stingency | Polynucleotide | Length | Hybridization Temperature | Temperature and |
| Condition | Hybrid | (bp)^{‡} | and Buffer^{†} | Buffer^{†} |
| A | DNA:DNA | ≧50 | 65° C.; 1xSSC -or- | 65° C.; 0.3xSSC |
| | | | 42° C.; 1xSSC, 50% formamide | |
| B | DNA:DNA | <50 | T_{B}*; 1xSSC | T_{B}*; 1xSSC |
| C | DNA:RNA | ≧50 | 67° C.; 1xSSC -or- | 67° C.; 0.3xSSC |
| | | | 45° C.; 1xSSC, 50% formamide | |
| D | DNA:RNA | <50 | T_{D}*; 1xSSC | T_{D}*; 1xSSC |
| E | RNA:RNA | ≧50 | 70° C.; 1xSSC -or- | 70° C.; 0.3xSSC |
| | | | 50° C.; 1xSSC, 50% formamide | |
| F | RNA:RNA | <50 | T_{F}*; 1xSSC | T_{F}*; 1xSSC |
| G | DNA:DNA | ≧50 | 65° C.; 4xSSC -or- | 65° C.; 1xSSC |
| | | | 42° C.; 4xSSC, 50% formamide | |
| H | DNA:DNA | <50 | T_{H}*; 4xSSC | T_{H}*; 4xSSC |
| I | DNA:RNA | ≧50 | 67° C.; 4xSSC -or- | 67° C.; 1xSSC |
| | | | 45° C.; 4xSSC, 50% formamide | |
| J | DNA:RNA | <50 | T_{J}*; 4xSSC | T_{J}*; 4xSSC |
| K | RNA:RNA | ≧50 | 70° C.; 4xSSC -or- | 67° C.; 1xSSC |
| | | | 50° C.; 4xSSC, 50% formamide | |
| L | RNA:RNA | <50 | T_{L}*; 2xSSC | T_{L}*; 2xSSC |
| M | DNA:DNA | ≧50 | 50° C.; 4xSSC -or- | 50° C.; 2xSSC |
| | | | 40° C.; 6xSSC, 50% formamide | |
| N | DNA:DNA | <50 | T_{N}*; 6xSSC | T_{N}*; 6xSSC |
| O | DNA:RNA | ≧50 | 55° C.; 4xSSC -or- | 55° C.; 2xSSC |
| | | | 42° C.; 6xSSC, 50% formamide | |
| P | DNA:RNA | <50 | T_{P}*; 6xSSC | T_{P}*; 6xSSC |
| Q | RNA:RNA | ≧50 | 60° C.; 4xSSC -or- | 60° C.; 2xSSC |
| R | RNA:RNA | <50 | T_{R}*; 4xSSC | T_{R}*; 4xSSC |
| ^{‡}The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity. | | | | |
| ^{†}SSPE (1xSSPE is 0.15 M NaCl, 10 mM NaH₂PO₄, and 1.25 mM EDTA, pH 7.4) can be substituted for SSC (1xSSC is 0.15 M NaCl and 15 mM sodium citrate) in the hybridization and wash buffers; washes are performed for 15 minutes after hybridization is complete. | | | | |
| *T_{B}-T_{R}: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) | | | | |
| # = 2(# of A + T bases) + 4(# of G + C bases). For hybrids between 18 and 49 base pairs in length, Tₘ(° C.) = 81.5 + 16.6(log₁₀[Na⁺]) + 0.41(% G + C) − (600/N), where N is the number of bases in the hybrid, and [Na⁺] is the concentration of sodium ions in the hybridization buffer ([Na⁺] for 1xSSC = 0.165 M). | | | | |

It will be clear to one of skill in the art that the contacting, hybridization and wash steps can be optimized using any suitable method of optimization established in the art. These include, but are not limited to, techniques that increase the efficiency of annealing or hybridization from complex mixtures of polynucleotides (e.g., PERT; Nucleic Acids Research 23:2339-2340, 1995) or hybridization in different formats (e.g., using an immobilized template or using microtiter plates; Analytical Biochemistry 227:201-209, 1995).

Polymorphisms can also be identified by hybridization to nucleic acid arrays, some examples of which are described in WO 95/11995. The same arrays or different arrays can be used for analysis of characterized polymorphisms. WO 95/11995 also describes subarrays that are optimized for detection of a variant form of a precharacterized polymorphism. Such a subarray contains probes designed to be complementary to a second reference sequence, which is an allelic variant of the first reference sequence. The second group of probes is designed by the same principles as described, except that the probes exhibit complementarity to the second reference sequence. The inclusion of a second group (or further groups) can be particularly useful for analyzing short subsequences of the primary reference sequence in which multiple mutations are expected to occur within a short distance commensurate with the length of the probes (e.g., two or more mutations within 9 to 21 bases).

Amplification products generated using the polymerase chain reaction (PCR) can be analyzed by the use of, for example, denaturing gradient gel electrophoresis (DGGE). Different alleles can be identified based on the different sequence-dependent melting properties and electrophoretic migration of DNA in solution. Erlich, ed., PCR Technology, Principles and Applications for DNA Amplification, (W.H. Freeman and Co, New York, 1992), Chapter 7.

Alleles of target sequences can be differentiated using single-strand conformation polymorphism analysis, which identifies base differences by detecting altered electrophoretic mobility of single-stranded PCR products (Orita et al., 1989. Proc. Nat. Acad. Sci. 86:2766-2770). Amplified PCR products can be generated as described above, and heated or otherwise denatured, to form single-stranded amplification products. Single-stranded nucleic acids may re-fold or form secondary structures that are partially dependent on the base sequence, such conformations leading to altered electrophoretic mobility. The different electrophoretic mobilities of single-stranded amplification products can be related to base-sequence differences between alleles of target sequences.

An alternative method for identifying and analyzing polymorphisms, for example, is based on single-base extension (SBE) of a fluorescently-labeled primer coupled with fluorescence resonance energy transfer (FRET) between the label of the added base and the label of the primer. Typically, the method, such as that described by Chen et al., (Proc. Natl. Acad. Sci. USA, 94:10756-61 (1997)), uses a locus-specific oligonucleotide primer labeled on the 5′ terminus with 5-carboxyfluorescein (FAM), or another suitable fluorophor. This labeled primer is designed so that the 3′ end is immediately adjacent to the polymorphic site of interest. The labeled primer is hybridized to the locus, and SBE of the labeled primer is performed with fluorescently-labeled dideoxyribonucleotides (ddNTPs), labeled with a fluorophor that is a FRET partner of the fluorophor used to label the primer. An increase in fluorescence of the added ddNTP in response to excitation at the wavelength of the labeled primer is used to infer the identity of the added nucleotide. Other suitable methods will be readily apparent to one of skill in the art.

In cases where polymorphisms affect the amino acid sequence of a gene product, the determination of the allele present in a sample obtained from an individual is made using techniques for protein detection. For example, antibodies that specifically interact with the allele-specific protein or polypeptide will be indicative of the specific allele present in the sample. The specific binding of such antibodies to protein or polypeptide gene expression products can be detected and measured by methods known in the art.

The detection of a particular allele by any of the methods described herein in a sample derived from an individual is indicative of the individual's susceptibility to atopy-associated diseases. Particular alleles at a polymorphic site that is linked to one or more atopy-associated diseases can be indicative of an increased probability (likelihood) of being susceptible to one or more atopy-associated disease. Alternatively, a different allele at the same or a different polymorphic site can be indicative of a decreased probability of being susceptible to one or more atopy-associated disease. For example, detection of one or more alleles at one or more polymorphic sites listed in Table 1 in a sample obtained from an individual is indicative of the individual's susceptibility to atopy-associated diseases. Detection of other alleles at one or more of the polymorphic sites listed in Table 1 in a sample from an individual would then be indicative of a decreased susceptibility to atopy-associated diseases relative to an individual possessing one or more of the alleles listed in Table 1.

In addition to methods of detecting SNPs as diagnostic methods for atopy-associated disease, the invention is directed to using particular alleles as therapeutic targets for atopy-associated diseases in the cases where the particular allelic versions are functionally responsible for effecting the disease phenotype. For example, polymorphisms that have direct functional consequences on a gene product (e.g., alteration of amino acid sequence, mis-regulation of a gene, alteration of a splice site, etc.) or the levels of a gene product can be the target of directed therapies to alleviate either the functional or regulatory consequences of the allele present at the particular polymorphic site.

The invention further provides kits comprising at least one allele-specific indicator as described herein. Often, the kits contain one or more pairs of allele-specific oligonucleotides hybridizing to different alleles at a polymorphic site. In some kits, the allele-specific oligonucleotides are provided immobilized to a substrate. For example, the same substrate can comprise allele-specific oligonucleotide probes for detecting at least 1, 5, 10 or all of the polymorphisms described herein. Optional additional components of the kit include, for example, restriction enzymes, reverse-transcriptase or polymerase, the substrate nucleoside triphosphates, means used to label (for example, an avidin-enzyme conjugate and enzyme substrate and chromogen if the label is biotin), and the appropriate buffers for reverse transcription, PCR, or hybridization reactions. Usually, reagents of the kit are packaged together with instructions for carrying out the methods.

The invention further relates to an oligonucleotide microarray having immobilized thereon a plurality of oligonucleotides that serve as allele-specific probes. For example, the microarray can contain one or more allele-specific probes for an allele at a polymorphic site listed in Table 1. The nucleic acid sequence surrounding the polymorphic sites listed in Table 1 can be used to design suitable oligonucleotide probes, and the preparation of such oligonucleotide microarrays is well known in the art.

The invention will be further described with reference to the following non-limiting examples. The teachings of all the patents, patent applications and all other publications and websites cited herein are incorporated by reference in their entirety.

### EXEMPLIFICATION

### Example 1

Families from the Saguenay-Lac-St-Jean region were recruited through media advertisement print and radio) and specialized clinics. Probands were required to fulfill at least two of the following three criteria: 1) a minimum of three clinic visits for acute asthma within one year; 2) two or more asthma-related hospital admissions within one year; and 3) steroid dependency, as defined by either 6 months of oral, or one year of inhaled, corticosteroid use. Families were included for study if at least one parent was available for assessment, one parent was unaffected, and all four grandparents were of French Canadian descent. Two sets of families were collected. The first included families with a minimum of two affected individuals (linkage families), and the second included families with only one affected individual (TDT families).

In total, 66 families were genotyped with respect to 42 polymorphic markers selected from 7 genomic regions previously identified as asthma/atopy candidate regions (Ober, C. et al., 1998. Hum. Mol. Genet., 7:1393-1398; Daniels, S. et al., 1996. Nature, 383:247-250; The CSGA, 1997. Nat. Genet., 15:389-392; Wjst, M. et al., 1999. Genomics, 58:1-8; Dizier, M. et al., 2000. Am. J Resp. Crit. Care Med., 162:1812-1818). Evidence suggestive of linkage to the atopy phenotype was demonstrated for chromosome 12 at marker D12S1052 (NPL=2.21, P=0.014). No evidence replicating linkage to any of the assessed phenotypes in the remaining chromosomal regions was obtained. Fine mapping on chromosome 12 was performed with 28 additional markers in a total of 121 families. Peak evidence for linkage was again demonstrated at the D12S1052 locus (NPL=2.27, p=0.013). Markers GATA71A08 and D12S1064, spanning a genetic distance of 18.5 cM, define the boundaries of this peak. An attempt was made to identify a disease haplotype that included (or was adjacent to) the D12S1052 locus, by genotyping six additional markers surrounding the D12S1052, and examining for transmission-disequilibrium. In the initial cohort of 121 families, a 2 locus haplotype D12S828*291-D12S869*226 exhibited significant transmission-disequilibrium (transmitted to untransmitted allele ratio 62:29, p=0.0019). This haplotype was further extended to incorporate the D12S376 locus. The 3 locus haplotype D12S376*152-D12S828*291-D12S869*226 also demonstrated significant distortion (34:12, p=0.002).

Comparative sequencing of candidate genes for atopy was performed in a subset from our cohort of 23 severely affected asthmatics. Using the GenBank and the Human Genome Browser databases, potential candidate genes from the 18 cM region demonstrating the strongest linkage for the atopy phenotype were identified. Attention was initially focused on 10 genes that have either been implicated in immune-related or inflammatory processes, smooth muscle contraction, or genes with significant expression in either lung tissue or inflammatory cells. All coding regions of the 10 candidate genes were fully re-sequenced, including all exon-intron junctions. Table 1 shows identified polymorphisms. A total of 20 polymorphisms were identified in seven genes, 7 of which were previously described (indicated by an asterisk). Nine polymorphisms were located within coding regions, one (CPM c1801T>C) was identified in a 3′ UTR region, and the remaining 10 were intronic. Of the nine polymorphisms located within coding regions, two were non-synonymous: Stem Cell Factor (SCF) c628G>T (Asp210Tyr) and IRAK-M c439G>A (Val147Ile). The remaining 6 polymorphisms were silent. In the cohort described herein, the c628 T allele of SCF was undertransmitted to asthmatics from their heterozygous parents (2:7), suggesting that this polymorphism has a protective role in the pathobiology of asthma and allergy. The allele frequency of this polymorphism was too low to detect significant levels of transmission disequilibrium.

TABLE 1
SNPs identified

| | | Accession | | | Amino Acid |
|---|---|---|---|---|---|
| Gene | Symbol | Number | Location | SNP | Change |
| Nucleosome | NAP1 | NM_004537 | Intron 2 | c206+6 G>A | non-coding |
| Associated Protein | | | | c206+14 A>G | non-coding |
| | | | | c206+105 T>C | non-coding |
| | | | Exon 3 | c300 C>T* | synonymous |
| | | | Intron 3 | c348+45 A>G | non-coding |
| | | | | c348+57 C>T | non-coding |
| | | | Intron 4 | c429+39 C>T | non-coding |
| | | | Exon 5 | c471 A>G* | synonymous |
| Carboxypeptidase M | CPM | NM_001874 | Intron 2 | c258−95 G>A | non-coding |
| | | | Exon 7 | c837 G>A | synonymous |
| | | | Exon 8 | c1077 T>C | synonymous |
| | | | 3′UTR | c1758 T>C* | non-coding |
| | | | 3′UTR | c1801 T>C | non-coding |
| Potassium Channel, | KCNMB4 | NM_014505 | Intron 2 | c336+70 A>G* | non-coding |
| Calcium-Activated, | | | | | |
| Large Conductance, | | | | | |
| Subfamily M, Beta | | | | | |
| Member 4 | | | | | |
| Stem Cell Factor | SCF | NM_000899 | Exon 7 | c628 G>T^{##} | Asp210Tyr |
| Interleukin Receptor | IRAK-M | NM_007199 | Intron 1 | c133−71 T>A | non-coding |
| Associated Kinase | | | Exon 4 | c435 A>G | synonymous |
| | | | Exon 5 | c439G>A* | Val147Ile |
| Signal Transduction | STAT6 | NM_003153 | Intron 11 | c1089+29 G>A | non-coding |
| and Activator of | | | | | |
| Transcription 6 | | | | | |
| Rattus potassium | POCHA^{#} | | Exon 2 | c321 C>T | synonymous |
| channel homologue | | | | | |

*Denoted SNPs previously described and listed on dbSNP: NAP c300C/T rs1059136; NAP c471A/G rs1059143; CPM c1758T/C rs1144947; KCNMB4 c336+70A/G rs710651; IRAK-M c439G/A rs1152888.
^{#}POCHA is a Rattus potassium homolog.
^{##}SCF c628 G>T was previously described (Heinzmann A. et al., 2000. Clin. Exp. Allergy, 30: 1555-1561).

Taken together, these findings provide evidence that these polymorphisms on chromosome 12 contribute to the pathobiology of atopy.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for determining an individual's susceptibility to atopy-associated disease, comprising detecting in a biological sample obtained from the individual an allele at a polymorphic site in the genetic region defined by the D12S376*152-D12S828*291-D12S869*226 haplotype, wherein said allele is identified as being linked to the atopy-associated disease phenotype.

2. The method of claim 1, wherein the atopy-associated disease is asthma.

3. The method of claim 1, wherein the method of detecting the allele comprises using an allele-specific indicator.

4. The method of claim 3, wherein the method of detecting the allele comprises a hybridization assay, and the allele-specific indicator is one or more allele-specific hybridization probe.

5. The method of claim 4, wherein the one or more allele-specific hybridization probes are contained in a microarray.

6. The method of claim 3, wherein the allele-specific indicator is an allele-specific antibody.

7. A method for determining an individual's susceptibility to atopy-associated disease, comprising detecting in a biological sample obtained from the individual one or more alleles at one or more polymorphic sites, said one or more alleles selected from the group consisting of: NM_{—}004537 c206+6 G>A, NM_{—}004537 c206+14 A>G, NM_{—}004537 c206+105 T>C, NM004537 c300 C>T, NM_{—}004537c348+45 A>G, NM_{—}004537 c348+57 C>T, NM_{—}004537 c429+39 C>T, NM_{—}004537 c471 A>G, NM_{—}001874 c258-95 C>A, NM_{—}001874 c837 G>A, NM_{—}001874 c1077 T>C, NM_{—}001874c1758 T>C, NM_{—}014505 c336+70 A>G, NM_{—}000899 c628 G>T, NM_{—}007199 c133-71 T>A, NM_{—}007199 c435 A>G, NM_{—}007199 c439 G>A, NM_{—}003153 c1089+29 G>A, and POCHA c321 C>T, and wherein detection of the allele is indicative of the individual's susceptibility to atopy-associated disease.

8. The method of claim 7, wherein the atopy-associated disease is asthma.

9. The method of claim 7, wherein the individual is of French-Canadian descent.

10. The method of claim 7, wherein the method of detecting the allele comprises using an allele-specific indicator.

11. The method of claim 10, wherein the method of detecting the allele comprises a hybridization assay, and the allele-specific indicator is one or more allele-specific hybridization probe.

12. The method of claim 11, wherein said one or more allele-specific hybridization probes are contained in a microarray.

13. The method of claim 7, wherein said allele is NM_{—}000899 c628 G>T or NM_{—}007199 c439 G>A.

14. The method of claim 13, said allele is detected using an allele-specific antibody.

15. A method for determining an individual's susceptibility to atopy-associated disease, comprising detecting the D12S376*152-D12S828*291-D12S869*226 haplotype, wherein the presence of the haplotype is indicative of an increased susceptibility to atopy-associated disease.

16. The method of claim 15, wherein the atopy-associated disease is asthma.

17. The method of claim 15, wherein the method of detecting the allele comprises a hybridization assay utilizing one or more allele-specific hybridization probes.

18. A method for determining an individual's susceptibility to atopy-associated disease, comprising detecting the D12S828*291-D12S869*226 haplotype, wherein the presence of the haplotype is indicative of an increased susceptibility to atopy-associated disease.

19. The method of claim 18, wherein the atopy-associated disease is asthma.

20. The method of claim 18, wherein the method of detecting the allele comprises a hybridization assay utilizing one or more allele-specific hybridization probes.

21. A nucleic acid molecule comprising an allele selected from the group consisting of: NM_{—}004537 c206+6 G>A, NM_{—}004537 c206+14 A>G, NM_{—}004537 c206+105 T>C, NM_{—}004537c348+45 A>G, NM_{—}004537 c348+57 C>T, NM_{—}004537 c429+39 C>T, NM_{—}001874 c258-95 C>A, NM_{—}001874 c837 G>A, NM_{—}001874 c1077 T>C, NM_{—}007199 c133-71 T>A, NM_{—}007199 c435 A>G, NM_{—}003153 c1089+29 G>A, and POCHA c321 C>T.

22. A kit for determining an individual's susceptibility to atopy-associated disease comprising reagents for determining one or more alleles at one or more polymorphic sites selected from the group consisting of: NM_{—}004537 c206+6, NM_{—}004537 c206+14, NM_{—}004537 c206+105, NM_{—}004537 c300, NM_{—}004537c348+45, NM_{—}004537 c348+57, NM_{—}004537 c429+39, NM_{—}004537 c471, NM_{—}001874 c258-95, NM_{—}001874 c837, NM_{—}001874 c1077, NM_{—}001874c1758, NM_{—}014505 c336+70, NM_{—}000899 c628, NM_{—}007199 c133-71, NM_{—}007199 c435, NM_{—}007199 c439, NM_{—}003153 c1089+29, and POCHA c321.

23. The kit of claim 22, wherein the kit comprises an allele-specific indicator.

24. The kit of claim 23, wherein the allele-specific indicator is an allele-specific probe.

25. The kit of claim 23, wherein the allele-specific indicator is an allele-specific antibody.

26. An oligonucleotide microarray having immobilized thereon a plurality of probes, wherein at least one of the probes is specific for an allele at a polymorphic site selected from the group consisting of: NM_{—}004537 c206+6, NM_{—}004537 c206+14, NM_{—}004537 c206+105, NM_{—}004537 c300, NM_{—}004537c348+45, NM_{—}004537 c348+57, NM_{—}004537 c429+39, NM_{—}004537 c471, NM_{—}001874 c258-95, NM_{—}001874 c837, NM_{—}001874 c1077, NM_{—}001874c1758, NM_{—}014505 c336+70, NM_{—}000899 c628, NM_{—}007199 c133-71, NM_{—}007199 c435, NM_{—}007199 c439, NM_{—}003153 c1089+29, and POCHA c321.

27. A method for determining an individual's susceptibility to atopy-associated disease comprising:
obtaining a biological sample from the individual; and
determining the amino acid present at one or more of amino acid position 147 of the Interleukin Receptor Associated Kinase and amino acid position 210 of Stem Cell Factor,
wherein presence of one or more of an isoleucine at amino acid position 147 of the Interleukin Receptor Associated Kinase or a tyrosine at amino acid position 210 of Stem Cell Factor is indicative of increased likelihood of an atopy-associated disease in the individual as compared with an individual having a valine at amino acid position 147 of the Interleukin Receptor Associated Kinase or an aspartate at amino acid position 210 of Stem Cell Factor, respectively.

28. A method of claim 27, wherein the amino acid present at both position 147 of the Interleukin Receptor Associated Kinase and position 210 of Stem Cell Factor is determined.

29. The method of claim 27, wherein the atopy-associated disease is asthma.
